(19) United States
(12) Patent Application Publication
(10) Pub. No.: US 2026/0174708 A1
(43) Pub. Date: Jun. 25, 2026

(54) MYO-INOSITOL AND THE PREVENTION OF ACCELERATED GROWTH

(71) Applicant: SOCIÉTÉ DES PRODUITS NESTLÉ S.A., Vevey (CH)

(72) Inventors: José Manuel RAMOS NIEVES, Lausanne (CH); Jaz LYONS-REID, Auckland (NZ); Aristea BINIA, Fribourg (CH); Wayne S. CUTFIELD, Auckland (NZ); Keith Malcolm GODFREY, Hampshire (GB); Shiao-Yng CHAN, Singapore (SG)

(21) Appl. No.: 19/128,422

(22) PCT Filed: Nov. 9, 2023
(86) PCT No.: PCT/EP2023/081266

§ 371 Date: May 8, 2025

(30) Foreign Application Priority Data
Nov. 11, 2022 (EP) 22206824.9

(51) Int. Cl. A61K 31/047; A61K 9/00; A61K 31/4415; A61K 31/525; A61K 31/593; A61K 31/714; A61K 35/745; A61K 35/747

(52) U.S. Cl. CPC A61K 31/047; A61K 9/0095; A61K 31/4415; A61K 31/525; A61K 31/593; A61K 31/714; A61K 35/745; A61K 35/747

(57) ABSTRACT

The present invention relates to compositions for use in the prevention of accelerated growth in an offspring, said composition comprising myo-inositol, a combination of Lactobacillus and Bifidobacterium and at least one vitamin selected from the group consisting of vitamin B2, vitamin B6, vitamin B12, vitamin D and mixtures thereof, and wherein the composition is administered to the mother of said offspring during preconception, and/or pregnancy and/or during lactation periods. The present invention further relates to a kit of part for preventing accelerated growth in an offspring.

### FIELD OF THE INVENTION

The present invention relates to compositions for use in the prevention of accelerated growth in an offspring, said composition comprising myo-inositol, a combination of Lactobacillus and Bifidobacterium and at least one vitamin selected from the group consisting of vitamin B2, vitamin B6, vitamin B12, vitamin D and mixtures thereof, and wherein the composition is administered to the mother of said offspring during preconception, and/or pregnancy and/or lactation periods. The present invention further relates to a kit of part for preventing accelerated growth in an offspring.

### BACKGROUND OF THE INVENTION

Generally, it is recommended that women take good care of themselves during pregnancy to prevent complications. Taking good care should include ensuring an optimal nutrition.

In general, scientific evidence is accumulating that prenatal nutrition causes programming of long-term growth, development and health and can impact the risk of developing chronic diseases. Several studies have shown that changes in dietary intake or manipulation of individual macro and micronutrients during preconception and pregnancy periods can have an impact in several physiological processes for the offspring, such as growth, metabolism, appetite, cardiovascular function among others (Koletzko B et al (2011) Am J Nutr 94 (s): 2036-43S).

Therefore, nutritional status (nutrient stores and dietary intake) and metabolic status of women before and during pregnancy and during lactation is of relevance to optimize neonatal and child health outcomes. Maternal nutrition is thought to affect the availability and supply of nutrients to the developing foetus that are required for critical developmental processes and examples of prenatal nutrient supplementation impact on postnatal growth of the offspring have been published (Lu W P et al. PLOS One. 2014:9.2; Bergmann R L et al. Journal of perinatal medicine. 2012:40 (6): 677-684).

A large variety of nutrients have already been used in compositions for maternal administration and various nutritional compositions have been developed to address maternal nutrition needs. These typically contain vitamin and mineral mixes.

However, it would still be useful to specifically target the nutritional status of this specific population by selecting nutrient supplementation tailored to the nutritional needs of women during preconception, pregnancy and lactation periods for the benefit of the mother and her offspring.

Normal growth is the progression of changes in height, weight and head circumference that are compatible with established standard for a given population. Normal growth is a reflection of overall health and nutritional status. Most healthy infants and children grow in a predictable fashion, and steadily within normal percentile ranges on standard growth curves. Occasionally, growth deviates in weight, height or head size and an undesirable rapid deviation from the expected growth trajectory could negatively affect future development and health. A growth problem means that an infant or a child fall either below or above the average range of growth for an infant's or a child's age, sex, family history or racial background. It has been demonstrated that growth in early life can affect long-term health. In particular, early postnatal growth acceleration increases long-term risk of obesity (Druet C et al. Paediatric and perinatal epidemiology. 2012: 26.1:19-26; Singhal A. Ann Nutr Metab. 2017: 70: 236-240) and is associated with risk factors for both cardiovascular outcomes and metabolic outcomes among preschool children (Li N. et L. Asia Pac J Clin Nutr. 2020: 29 (3): 558-565). Importantly, the accelerated growth phenomenon could be observed in healthy children, who because of genetic predisposition or due to nutritional factors, experience an accelerated increase in weight or length postnatally, but it can also be observed in those children that suffered a level of growth faltering due to pre- or post-natal insults of nutritional nature (Singhal A. Ann Nutr Metab. 2017:70: 236-240).

Nutrition is one factor among others that affect growth and development in infants. However, no maternal nutritional intervention has been developed to specifically contribute to the prevention of accelerated growth in early life or childhood. It would therefore be desirable to have such intervention available.

Further, a combination of optimal nutrition before and/or during pregnancy and/or during lactation periods with a composition that can contribute to the prevention of accelerated growth in an offspring would be desirable as well.

The objective of the present invention was hence to enrich or improve the state of the art and in particular, to provide a maternal composition for use in the prevention of accelerated growth in an offspring.

The publications discussed herein are provided solely for their disclosure prior to the filing date of the present application. Nothing herein is to be construed as an admission that such publications constitute prior art to the claims appended hereto.

### SUMMARY OF THE INVENTION

The object of the present invention is achieved by the subject matter of the independent claims. The dependent claims further develop the idea of the present invention.

The inventors have found that a composition comprising myo-inositol, a combination of probiotics and other nutrients administered to a mother during preconception, and/or pregnancy and/or lactation periods may have beneficial effects in preventing accelerated growth her offspring.

Thus, in a first aspect the present invention provides a composition for use in the prevention of accelerated growth in an offspring, said composition comprising myo-inositol, a combination of Lactobacillus and Bifidobacterium probiotics and at least one vitamin selected from the group consisting of vitamin B2, vitamin B6, vitamin B12, vitamin D and mixtures thereof and wherein said composition is administered to the mother of said offspring during preconception, and/or pregnancy and/or lactation periods.

The invention further provides a kit of parts comprising at least two physically separated compositions, each composition comprising at least one ingredient selected from the group consisting of vitamin B2, vitamin B6, vitamin B12 and vitamin D, at least one of the separated compositions comprises myo-inositol and at least one of the separated compositions comprises a combination of Lactobacillus and Bifidobacterium probiotics, for use in the prevention of accelerated growth in an offspring, wherein the two physically compositions are administered to the mother of said offspring during preconception, and/or pregnancy and/or lactation periods.

### DETAILED DESCRIPTION OF SOME EMBODIMENTS OF THE INVENTION

### Definitions

Unless defined otherwise, all technical and scientific terms used herein have the same meaning as commonly understood by one of ordinary skill in the art.

The term “infant” means a human subject under the age of 12 months or an age equivalent non-human animal.

The terms “young child” or “toddler” as used herein may mean a human subject aged between 12 months and 5 years of age. Suitably, a “young child” may refer to an age equivalent non-human animal.

The expression “later in life” includes childhood and adulthood. For example, “later in life” may refer to childhood, such as to an age of at least 1 year, 2 years, at least 3 years, at least 4 years, or at least 5 years. “Later in life” may refer to adolescent period or young adulthood.

The term “subject” as used herein refers to a mammal and more particularly a cat, a dog or a human. The human may be a woman, for example, a woman who wants to get pregnant, a pregnant woman or a lactating woman. In an embodiment of the invention the subject is a mammal selected from the group consisting of a cat, a dog and a human.

For example, the subject may a woman who is trying to get pregnant, or a pregnant woman or a lactating woman.

Within the context of the present invention, the expression “accelerated growth” refers to a rapid infant or young child weight or length or body mass index (BMI) gain and it is defined as an increase in weight or length or BMI of 0.67 standard deviations (SD) scores or more between birth and 1 year of age or birth and 2 years of age or birth and 5 years of age, which is equivalent to upwards crossing of one or more percentile band on standard growth charts (e.g. World Health Organization Child Growth Standards), that is second to nineth percentile, nineth to 25^{th} percentile, 25^{th} to 50^{th} percentile, and so on.

BMI is a person's weight in kilograms (or pounds) divided by the square of height in meters (or feet). A high BMI can indicate high body fatness. The risk of accelerated growth was assessed from birth to 1 year and from birth to 2 years.

Within the context of the present invention, the expression “nutritional composition” refers to a composition which nourishes a subject. This nutritional composition is usually taken orally or intravenously. It may include a lipid or fat source, a carbohydrate source and/or a protein source.

Within the context of the present invention, the term “nutrient” or “nutrients” is intended to comprise both macronutrients (for example carbohydrates, proteins or fats) and micronutrients (for example minerals or vitamins) and components thereof (e.g., fatty acids, amino acids) for the human body.

Within the context of the present invention, the terms “ingredient” or “ingredients” indicate an edible substance or mixture of substances which comprise or is essentially consisting of a nutrient for the human body.

Within the context of the present invention, the expressions “in the prevention of”, “to prevent” or “for the prevention of” can be used interchangeably. They should be understood as comprising the decrease of the severity of a disease process. These expressions also encompass the relieve of the symptoms of induced by a disease, such as stress and/or decrease of complications caused by the disease.

As used in the specification, the term “comprise” or “comprises” or “comprising” and the like are to be construed in an inclusive sense as opposed to an exclusive or exhaustive sense; that is to say, in the sense of “including”, but not “limited to”.

As used herein, “about” is understood to refer to numbers in a range of numerals, for example the range of −10% to +10% of the referenced number, within −5% to +5% of the referenced number, or in one aspect, within −1% to +1% of the referenced number, and in a specific aspect, within −0.1% to +0.1% of the referenced number. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

The terms “in particular” or “more particularly” as used herein should not be considered limiting but should be interpreted as being synonymous with “for example” or “especially”.

As used in the specification, the singular forms “a”, “an” and “the” include plural referents unless the context clearly dictate otherwise. Thus, for example, reference to “a component” or “the component” includes two or more components.

All percentages are by weight unless otherwise stated.

Unless defined otherwise, all technical and scientific terms have and should be given the same meaning as commonly understood by one of the ordinary skills in the art which this invention belongs.

The term dry weight refers to the measurement of the mass of matter when completely dried and all fluids removed form the matter. The dry weight % of a substance refers to the relative amount of said substance in the total dry matter. As used in the specification, the expression “weight %” and “wt %” are synonymous.

It is noted that the various features, aspects, examples and embodiments described in the present invention may be compatible and/or combined together.

Other definitions and/or explanations are present in the detailed description below.

### EMBODIMENTS OF THE INVENTION

The invention will now be described in further details.

In a first aspect, the present invention relates to a composition for use in the prevention of accelerated growth in an offspring, said composition comprising myo-inositol, a combination of Lactobacillus and Bifidobacterium probiotics and at least one vitamin selected from the group consisting of vitamin B2, vitamin B6, vitamin B12, vitamin D and mixtures thereof and wherein said composition is administered to the mother of said offspring during preconception, and/or pregnancy and/or lactation periods.

In an embodiment, the composition thus reduces risk of experiencing excess fat mass later in life, in particular it reduces risk of experiencing excess fat mass at 1 and 2 years of life by reducing the risk of accelerated growth from birth to one year of life as well as from birth to 2 years of life.

Advantageously, the composition according to the present invention is administered to the mother during preconception, and/or pregnancy period and/or lactation periods, thereby providing tailored nutrient supplementation allowing prevention of accelerated growth in her offspring.

Myo-Inositol, or cis-1,2,3,5-trans-4,6-cyclohexanehexol, is the predominant isomeric form of inositol. Myo-Inositol is a compound present in animal and plant cells and plays an important role in various cellular processes, as the structural basis for secondary messengers in eukaryotic cells, in particular as inositol triphosphates (IP3), phosphatidylinositol phosphate lipids (PIP2/PIP3) and inositol glycans. Myo-inositol has been shown to participate in a variety of biological process such as cell growth and survival, development and function of peripheral nerves, osteogenesis, energy metabolism and reproduction (Croze et al. Biochimie. 2013:95:1811-1827). Myo-inositol is found as free form, phosphoinositides and phytic acid, in fresh fruits and vegetables, and in all foods containing seeds (beans, grains and nuts) (Clements R S and Darnell B. Am J Clin Nutr. 1980:33:1954-1967). Myo-Inositol from phytic acid can be released in the gut by phytases found in plants, microorganisms and in animal tissues (Schlemmer U et al. Mol Nutr Food Res. 2009:53:5330-S375). These enzymes are capable of releasing free inositol, orthophosphate, and intermediary products including the mono-, di-, tri-, tetra- and penta-phosphate forms of inositol. Much of the ingested inositol hexaphosphate is hydrolysed to inositol. Myo-inositol is also commercially available from several suppliers.

Myo-Inositol may be administered in accordance with the present invention in any effective amount. Typically, an effective amount will depend on the type, age, size, health status, lifestyle and/or genetic heritage of the subject. The effective amount may be split into several smaller amounts and administered throughout the day so as the total daily intake is the effective amount. A person skilled in the art will be able to propose appropriate amounts of myo-inositol to be consumed per day. For example, the composition for use in accordance with the present invention may provide myo-inositol in an amount of 0.2 to 5 g, preferably 1.5 to 5 g, more preferably 2 to 5 g, most preferably 2 to 4 g per daily dose.

Suitably, the composition for use according to the present invention further comprises a combination of Lactobacillus and Bifidobacterium probiotics. It is particularly beneficial to combine myo-inositol with probiotics, as probiotics have been found to improve gut barrier function and to help nutrients pass through the gut (Cani P D et al. Gut. 2009: 58: 1091-1103). Combining myo-inositol with probiotics thus enhances the absorption of myo-inositol and other nutrients that may be present in the composition.

The term “probiotic” means microbial cell preparation or components of microbial cells with a beneficial effect on the health or well-being of the host (Salminen S, Ouwehand A. Benno Y. et al. “Probiotics: how should they be defined” Trends Food Sci. Technol. 1999:10 107-10). The microbial cells according to the present invention are generally bacteria. Within the context of the present invention the term probiotics refers to live probiotic bacteria, non-replicating probiotic bacteria, dead probiotic bacteria, non-viable probiotic bacteria, fragments of probiotic bacteria such as DNA, metabolites of probiotic bacteria, cytoplasmic compounds of probiotic bacteria, cell wall materials of probiotic bacteria, culture supernatants of probiotic bacteria, and/or combinations of any of the foregoing. The probiotic may for example be live probiotic bacteria, non-replicating probiotic bacteria, dead probiotic bacteria, non-viable probiotic bacteria, or any combination thereof. In one embodiment of the present invention the probiotic is live probiotic bacteria.

The term “cfu” should be understood as colony forming unit.

In some embodiments, the Lactobacillus strain is the Lactobacillus rhamnosus strain available under the deposit number CGMC 1.3724 and the Bifidobacterium is the Bifidobacterium lactis BB12 strain deposited as CNCM I-3446. Preferably, the composition comprises a combination of the Lactobacillus rhamnosus strain available under the deposit number CGMCC 1.3724 and of the Bifidobacterium lactis BB12 strain deposited as CNCM I-3446. Most preferably the combination of probiotics consists of a combination of the Lactobacillus rhamnosus strain available under the deposit number CGMCC 1.3724 and of the Bifidobacterium lactis BB12 strain deposited as CNCM I-3446. In a preferred embodiment, the probiotic is provided in an amount of from 10⁵ to 10¹² colony forming units (cfu) per daily dose, more preferably from 10⁷ to 10¹¹ cfu per daily dose.

Suitably, the composition for use according to the present invention further comprises a combination of vitamins. Suitably, the composition may comprise at least one vitamin selected from the group consisting of vitamin B2, vitamin B6, vitamin B12, vitamin D and mixtures thereof. For example, the composition comprises vitamin B2, vitamin B6, vitamin B12 and vitamin D. Preferably the vitamin D is vitamin D3.

The present inventors were able to show (Crozier S R et al., Am J Clin Nutr. 2012:96:57-63; Godfrey K M et al., Diabetes. 2011:60:1528-34; Childs C et al., J Dev Orig Health Dis. 2015:6 (Suppl 2): $36.) that pregnant women were more often deficient in vitamins B6, B12 and D compared to other nutrients. Also, they have demonstrated that vitamin B2 was not consumed in sufficient amounts by a significant proportion of the pregnant woman population. It is therefore of particular interest to supplement the diet of pregnant women with these vitamins in order to compensate these particularly often-occurring deficiencies.

If present, the vitamin B2 is preferably provided in an amount of from 0.14 to 14 mg per daily dose. If present, the vitamin B6 is preferably provided in an amount of from 0.19 to 19 mg per daily dose. If present, the vitamin B12 is preferably provided in an amount of from 0.26 to 26 μg per daily dose. If present, the vitamin D is preferably provided in an amount of from 1.5 to 100 μg per daily dose. Most preferably, the composition for use in accordance with the present invention comprises 1.8 mg of vitamin B2, 2.6 mg of vitamin B6, 5.2 μg of vitamin B12 and 10 μg of vitamin D per daily dose.

In some embodiments, the composition for use according to the present invention may comprise myo-inositol, vitamin B2, vitamin B6, vitamin B12, vitamin D, a combination of Lactobacillus rhamnosus strain available under the deposit number CGMC 1.3724 and Bifidobacterium lactis BB12 strain deposited as CNCM I-3446.

In some embodiments, the composition for use according to the present invention may comprise from 0.2 to 5 g of myo-inositol, from 0.14 to 14 mg of vitamin B2, from 0.19 to 19 mg of vitamin B6, from 0.26 to 26 μg of vitamin B12, from 1.5 to 100 μg of vitamin D, from 10⁵ to 10¹² cfu of Bifidobacterium lactis BB12 CNCMI-3446 and a from 10⁵ to 10¹² cfu of Lactobacillus rhamnosus CGMCC 1.3724, all amounts being defined by daily dose. Suitably, the offspring in which the accelerated growth is prevented may be an infant, a young child or a toddler.

Suitably, the composition for use according to the present invention prevents accelerated growth in an offspring during early life and/or childhood.

In some embodiments of the present invention, the accelerated growth occurs during the first 2 to 5 years of life of the offspring, for example during the first 2 to 4.5 years of life, or during the first 2 to 4 years of life, for example during the first 2 to 3.5 years of life. For example, the accelerated growth may occur in an offspring aged of 1 month, 2 months, 3 months, 6 months, 9 months, 12 months, 18 months, 2 years, 2.5 years, 3 years, 3.5 years, 4 years, 4.5 years or 5 years. In some embodiments, the prevention of accelerated growth may occur in an offspring of 5 years old, or of 4.5 years old, or of 4 years old, or of 3.5 years old, or of 3 years old, or of 2.5 years old, or of 2 years old, or of 18 months old, or of 12 months old, or of 9 months old, or of 6 months old, or of 3 months old, or of 2 months old, or age of 1 month old. The prevention of accelerated growth may occur in an offspring aged between 12 months and 5 years, for example aged between 18 months and 4.5 year, or aged between 2 and 3.5 years. In other embodiments, the accelerated growth may occur in an offspring up to 5 years of age, or up to 4.5 years of age, or up to 4 years of age, or up to 3.5 years of age, or up to 3 years of age, up to 2.5 years of age, or up to 2 years of age, or up to 18 years of age, or up to 12 months of age, or up to 9 months of age. In still another embodiment, the accelerated growth may occur in an offspring age between 1 months and 5 years.

The inventors have also provided evidence (U.S. Pat. No. 14,719) of common zinc deficiencies in at least certain populations of pregnant woman, even though in a lesser extent than vitamins B2, B12, B6 and D. Zinc is known to play a role in catalytic activities of various enzymes, in immune function, in protein synthesis, in DNA synthesis, in growth and development during pregnancy, childhood and adolescence and zinc insufficiency during pregnancy may lead to growth restrictions in the infants. It is therefore also particularly advantageous to supplement the diet of pregnant women with zinc. Thus, in some embodiments, the composition for use in accordance with the present invention may comprise zinc. Zinc may be present in an amount of from 1.1 to 40 mg per daily dose. Additional vitamins and minerals may also be added. For example, vitamins and minerals may be added in accordance with the recommendations of Government bodies such as the USRDA. For example, the composition may contain one or more of the following micronutrients, calcium, magnesium, phosphorus, iron, zinc, copper, iodine, selenium, vitamin A or retinol activity equivalent (RAE) for example in the form of beta carotene or a mix of carotenoids, Vitamin C, Vitamin B1, niacin, folic acid, biotin, Vitamin E. Preferably the composition may contain one or more of the following micronutrients in the following amounts: 100 to 2500 mg calcium, 35 to 350 mg magnesium, 70 to 3500 mg phosphorus, 2.7 to 45 mg iron, 1.1 to 40 mg zinc, 0.1 to 10 mg copper, 22 to 1,100 μg iodine, 6 to 400 μg selenium, 77 to 3000 μg of vitamin A or retinol activity equivalent (RAE) for example in the form of beta carotene or a mix of carotenoids, 8.5 to 850 mg Vitamin C, 0.14 to 14 mg Vitamin B1, 1.8 to 35 mg niacin, 60 to 1000 μg folic acid, 3 to 300 μg biotin, 1.9 to 109 μg Vitamin E.

The composition for use in accordance with the present invention may advantageously further comprise at least one oil selected from long chain polyunsaturated fatty acids, such as arachidonic acid (ARA), eicosapentaenoic acid (EPA) and/or docosahexaenoic acid (DHA), in any suitable amount as known by the person skilled in the art, for example in an amount of 100-500 mg per daily dose, more preferably between 200 and 400 mg per daily dose.

Any of the nutrients mentioned herein may be used in any amount that is effective in achieving the objective of the present invention. Skilled artisans will be able to determine appropriate dosages. Typically, dosage will depend on age, size and health status of the mother, on her lifestyle, as well as on her genetic heritage.

Typically, amounts are defined in the present application as amounts per daily dose. The amount of nutrient may thus be selected in each composition accordingly depending upon whether it is intended to be consumed once a day or more or less frequently.

The nutrients may be provided as a sustained release formulation. This way, the nutrient can be consumed less frequently, while the body is still constantly supplied with sufficient amount of such nutrient.

In some embodiments, the composition for use in accordance with the present invention by preventing the accelerated growth in the offspring further prevents development of metabolic disorders later in life in the offspring. Therefore, the present invention has beneficial effect for the mother and for the offspring, at early and late stage of his development.

Suitably, the metabolic disorders that may develop later in life are cardiometabolic disorders, obesity, excess fat mass deposition and overweight.

Cardiometabolic disorders may be hypertension, high triglyceridemia, high diastolic- or systolic blood pressure (Li N et al. Asia Pac J Clin Nutr. 2020:29 (3): 558-565)

“Obesity” is a condition in which the natural energy reserve, stored in the fatty tissue of animals, in particular humans and other mammals, is increased to a point where it is associated with certain health conditions or increased mortality. “Obese” is defined for an individual as having a BMI greater than 30. “Overweight” is defined for an individual as having a BMI over 25.

For children the BMI is plotted on a BMI vs. age growth standard chart (for either girls or boys) to obtain a percentile ranking. Percentiles are the most commonly used indicator to assess the size and growth patterns of individual children. The percentile indicates the relative position of the child's BMI among children of the same sex and age. Children and teenagers from 2 to 19 years of age, respectively, are considered overweight if their BMI is located between the 85^{th} and 95^{th} percentile. Children and teenagers from 2 to 19 years of age are considered obese if their BMI is located on or above the 95^{th} percentile or equal to the 95^{th} or an absolute BMI equal or greater to 30 kg/m2, whichever is lower.

Metabolic disorders that are associated with overweight, obesity and/or excessive fat accumulation are similar and well known to skilled artisans. For example, these disorders include cardiovascular diseases such as coronary heart disease; insulin resistance; type 2 diabetes; hypertension; sleep apnea, respiratory problems and/or dyslipidemia; but also some cancers such as endometrial, breast, and/or colon cancer; stroke; liver, non-alcoholic fatty liver and gallbladder disease; osteoarthritis; and/or gynecological problems.

Development of metabolic disorders associated with accelerated growth are typically observed in studies in early childhood, for example around the age of 5 years old, in adolescents and young adults (Li N et al. Asia Pac J Clin Nutr. 2020:29 (3): 558-565); Sutharsan R et al. Journal Health Population and Nutrition. 2015:33: 13). However, development of metabolic disorders associated with accelerated growth may start before 5 years of age, for example at 1, 2, 3 or 4 years old.

Suitably, the prevention of development of metabolic disorders later in life of the offspring can be observed in early childhood, for example around the age of 1 year, 2 years, 3 years, 4 years or 5 years.

In some embodiments, the composition for use according to the present invention may be intended for maternal administration. This means that the composition may be administered to female subject desiring to get pregnant, in particular during preconception period, to a pregnant female subject, in particular during pregnancy period or to a female subject who gave birth, in particular during lactation period.

In some embodiments, the female subject is a mammal, for example selected from the group consisting of a cat, a dog and a human.

Suitably, the female subject can be the mother of an offspring.

Suitably, the composition may be administered to a woman desiring to get pregnant and/or to a pregnant woman and/or to a lactating woman. Preferably, the composition for use in accordance with the present invention is to be administered to a woman desiring to get pregnant and/or to a pregnant woman, most preferably to a pregnant woman.

The inventors have found that administration of the composition according to the present invention to the mother of an offspring during preconception and/or during pregnancy and/or during lactation periods allows prevention of accelerated growth in her offspring. In some embodiments, the composition for use according to the present invention is administered to the mother of the offspring during preconception, and/or pregnancy and/or lactation periods. Preferably, the composition for use according to the present invention is administered to the mother of the offspring during preconception, and/or pregnancy periods.

In some embodiments, the composition for use according to the present invention may be administered to a mother during preconception period, for example, during at least 1, 2, 3 or 4 months preceding the pregnancy or desired pregnancy. Suitably, when the composition is to be administered to a mother during pregnancy, the composition is preferably administered for at least 4, for at least 8, for at least 12, for at least 16, for at least 20, for at least 24, for at least 28, for at least 36 weeks during pregnancy.

As the nutritional requirements increase in the second and third trimester of pregnancy, in some embodiments of the present invention, the composition for use according to the present invention is administered throughout the second and the third trimester pf pregnancy.

In some embodiments, the composition for use according to the present invention may be administered to a mother during lactation period, for example, during at least 1, 2, 3, 4, 5, 6, 7, 8, 9, 10, 11 or 12 months after delivery. Suitably, when the composition is to be administered to a mother during lactation period, the composition is administered for at 4, for at least 8, for at least 12, for at least 16, for at least 20, for at least 24, for at least 28, for at least 36 weeks during lactation.

It is important that women desiring to get pregnant prepare their body to the pregnancy by taking the appropriate nutrients since women's nutrients needs increase during pregnancy. The composition for use according to the present invention is specifically designed to meet the needs of women during these periods, for example before and during pregnancy, and just after delivery.

The composition may be in any form that is suitable to administer all the ingredients. Suitably, the composition for use according to the present invention may be in the form of a powdered nutritional composition that can be reconstituted in milk, water or any liquid, of a food product, of a drink, of a nutritional supplement or of a nutraceutical.

When the composition is in the form of a powdered nutritional composition to be reconstituted in milk or water it preferably comprises a protein source, a carbohydrate source and a lipid source, preferably together with lecithin. It may also comprise soya lecithin and/or a bulking agent. The protein source may be dried milk or dried skimmed milk. As carbohydrate source sucrose and/or maltodextrin may be used. The lipid source may be vegetable oil. The formulation may also alternatively or additionally contain glucose syrup, milk fat, magnesium citrate, choline salts and esters, prebiotic fibres, and/or ascorbyl palmitate. Flavor compounds, such as cocoa powder or honey, for example, may be added to provide taste variations.

The composition may be any type of composition suitable for consumption for the subject to whom it is to be administered.

The composition may also be a product selected from the group consisting of a nutritional product, a functional food product, a healthy ageing product, a dairy product, a dairy alternative product, a beverage product, a diet product, and a pet food product.

The term “nutritional product”, as used herein, means any product that can be used to provide nutrition to a subject. Typically, nutritional products contain a protein source, a carbohydrate source and a lipid source.

The term “food product”, as used herein, refers to any kind of product that may be safely consumed by a human or an animal. Said food product may be in solid, semi-solid or liquid form and may comprise one or more nutrients, foods or nutritional supplements. For instance, the food product may additionally comprise the following nutrients and micronutrients: a source of proteins, a source of lipids, a source of carbohydrates, vitamins and minerals. The composition may also contain antioxidants, stabilizers (when provided in solid form) or emulsifiers (when provided in liquid form).

The term “functional food product”, as used herein, refers to a food product providing an additional health-promoting or disease-preventing function to the individual.

The term “healthy ageing product”, as used herein, refers to a product providing an additional health-promoting or disease-preventing function related to healthy ageing to the individual.

The term “dairy products”, as used herein, refers to food products produced from milk or fractions of milk from animals such as cows, goats, sheep, yaks, horses, camels, and other mammals. Examples of dairy products are low fat milk (e.g. 0.1%, 0.5% or 1.5% fat), fat-free milk, milk powder, whole milk, whole milk products, butter, buttermilk, buttermilk products, skim milk, skim milk products, high milk-fat products, condensed milk, crème fraiche, cheese, ice cream and confectionery products, probiotic drinks or probiotic yoghurt type drinks.

The term “dairy alternative product” refers to products similar to dairy products but produced without milk.

The term “milk” is defined by Codex Alimentarius as the normal mammary secretion of milking animals obtained from one or more milkings without either addition to it or extraction from it, intended for consumption as liquid milk or for further processing.

The term “beverage product” as used herein, refers to a nutritional product in liquid or semi-liquid form that may be safely consumed by an individual.

The term “diet product” as used herein, refers to a food product with a restricted and/or reduced caloric content.

The term “pet food product” as used herein refers to a nutritional product that is intended for consumption by pets. A pet, or companion animal, as referenced herein, is to be understood as an animal selected from dogs, cats, birds, fish, rodents such as mice, rats.

The term “nutritional supplement” as used herein, refers to a nutritional product that provides nutrients to an individual that may otherwise not be consumed in sufficient quantities by said individual. For instance, a nutritional supplement may include vitamins, minerals, fiber, fatty acids, or amino acids. Nutritional supplements may for example be provided in the form of a pill, a tablet, a lozenge, a chewy capsule or tablet, a tablet or capsule, or a powder supplement that can for example be dissolved in water or sprinkled on food. Nutritional supplements typically provide selected nutrients while not representing a significant portion of the overall nutritional needs of a subject. Typically, they do not represent more than 0.1%, 1%, 5%, 10% or 20% of the daily energy need of a subject. A nutritional supplement may be used during preconception period or pregnancy or during lactation period, e.g., as a maternal supplement.

All ingredients of the composition can be admixed together or alternatively the composition can be provided in the form of a kit of parts wherein ingredients or groups of ingredients are provided separately. These separate compositions may be intended to be consumed separately or together.

In a further aspect, the present invention relates a kit of parts for use in the prevention of accelerated growth in an offspring, said kit of parts comprising at least two physically separated compositions each comprising at least one of the ingredients mentioned above, wherein at least one of the physically separated compositions comprises myo-inositol and at least one of the physically separated compositions comprises probiotics.

In some embodiments, it is provided a kit of parts comprising at least two physically separated compositions, each composition comprising at least one ingredient selected from the group consisting of vitamin B2, vitamin B6, vitamin B12 and vitamin D, at least one of the separated compositions comprises myo-inositol and at least one of the separated compositions comprises a combination of Lactobacillus and Bifidobacterium probiotics, for use in the prevention of accelerated growth in an offspring, wherein the two physically compositions are administered to the mother of said offspring during preconception, and/or pregnancy and/or lactation periods.

Each of the physically separated compositions can further comprise one or more of the ingredients mentioned above. Suitably, the first composition comprises at least one vitamin selected from the group consisting of vitamin B2, vitamin B6, vitamin B12, vitamin D and mixtures thereof and the second composition comprises probiotics. Myo-inositol can be provided in the first or the second composition, or even separately, but is preferably provided in the composition comprising the vitamins.

In one embodiment, the kit of parts comprises a first composition comprising myo-inositol and at least one vitamin selected from the group consisting of vitamin B2, vitamin B6, vitamin B12, vitamin D and mixtures thereof, optionally with other vitamins and/or nutrients mentioned above, except probiotics, and a second composition comprising a combination of Lactobacillus and Bifidobacterium probiotics.

In another embodiment, the kit of parts comprises a first composition comprising myo-inositol, vitamin B2, vitamin B6, vitamin B12, vitamin D, mixtures thereof, and zinc and a second composition comprising a combination of Lactobacillus and Bifidobacterium probiotics. Separation of probiotics from other ingredients is preferable to avoid any damage to the probiotics due to the presence of high concentration of minerals or other nutrients.

Suitably, the composition or the compositions comprised in the kit of parts may be administered regularly, for example once a day, two times a day, every two days or weekly.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the non-limiting examples.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the non-limiting examples.

### EXAMPLES

### Example 1

A nutritional supplement in powder form intended to be dissolved in water is provided with the ingredients as shown in Table 1 here below.

TABLE 1
nutritional supplement

| Ingredient | Amount per daily dose | |
|---|---|---|
| Myo-inositol | 4 | g |
| Vitamin D | 10 | μg |
| Vitamin B6 | 2.6 | mg |
| Vitamin B12 | 5.2 | μg |
| Vitamin B2 | 1.8 | mg |
| Zinc | 10 | mg |
| β-carotene | 720 | μg |
| Folic acid | 400 | μg |
| Iron | 12 | μg |
| Calcium | 150 | μg |
| Iodine | 150 | μg |

| | |
|---|---|
| Lactobacillus rhamnosus ¹⁾ | 1 × 10⁹ cfu |
| Bifidobacterium lactis BB12²⁾ | 1 × 10⁹ cfu |

¹⁾ Lactobacillus rhamnosus was deposited as CGMCC 1.3724.
²⁾Bifidobacterium lactis BB12 was deposited as CNCM I-3446.

The composition is provided as one sachet containing the probiotic and all other ingredients as a powder. The composition was administered to women desiring to get pregnant during at least one month prior to pregnancy and later to the same women until delivery.

### Example 2

Five hundred eighty-five women participating in the NiPPER trial (Godfrey K M et al; Trials (2017), 18:131.) across three clinical sites were included in the analyses. This included 295 in the intervention and 290 in the control group. On average, women were 30 years of age, at the upper end of the normal weight range, and predominantly nulliparous. European and Chinese ethnicities accounted for >80% of ethnicities. Participants were randomly allocated to receive twice-daily control nutritional powdered drink enriched with standard micronutrients, or twice-daily intervention nutritional powdered drink enriched with micronutrients, myo-inositol and probiotics, i.e., composition according to the present invention. The women who conceived within the first year of starting the nutritional powdered drink were followed through pregnancy and studied with their offsprings during the first two years of life.

The nutritional intervention led to a statistically significant reduction in the risk of experiencing accelerated growth (increase in >0.67 in weight-for-age z-scores/year) from birth to 1 year of age and from birth to 2 years of age in the offspring of mothers who received the composition according to the present invention. As observed in Table 2, 21.9% of the infants in the Intervention experienced accelerated growth from birth to 1 year of age compared to 31.1% in the Control group. This resulted in an adjusted risk ratio (aRR) of 0.76 for the offspring of intervened women, equivalent to a 24% reduction in the risk observed in the offspring of Control women. Similarly, 7.7% of the offspring of intervened women experienced accelerated growth from birth to 2 years of age, compared to 17.1% in the Control group. With an aRR of 0.55, there was a 45% reduction in the risk of experiencing accelerated growth during this time span.

TABLE 2
adjusted risk ratios (aRR) and 95% confidence interval (CI)
of rapid weight gain from birth to 1 and 2 years of age

| | n (%) | | aRR | |
|---|---|---|---|---|
| | Intervention | Control | (95% CI) | p-value |
| >0.67 SD from | 58 | 80 | 0.76 | 0.046 |
| birth to 1 year | (21.9%) | (31.1%) | (0.58, 0.99) | |
| >1.34 SD from | 19 | 43 | 0.55 | 0.014 |
| birth to 2 year | (7.7%) | (17.1%) | (0.34, 0.88) | |

Data are aRR and respective 95% CI from logistic regression adjusted for study site (UK/Singapore/New Zealand), infant sex (male/female), parity (nulliparous/multiparous), maternal smoking (none/active or passive), maternal pre-pregnancy BMI, age at visit and gestational age at birth. Statistically significant comparisons (p > 0.05) are shown in bold.

### Example 3

Accelerated growth during the first two years of life is associated to greater risk of obesity during childhood and adulthood (Druet C (2012) Paediatric and perinatal epidemiology 26.1:19-26). The nutritional intervention led to a statistically significant reduction in the risk of obesity at 2 years of age as defined by having a BMI >95^{th} percentile (BMI>1.65) in the offspring of the mother who received the composition according to the present invention (i.e. the intervention), as described in Table 3.

TABLE 3
adjusted risk ratios (aRR) and 95% confidence interval (CI)
of rapid weight gain from birth to 1 and 2 years of age

| | | Intervention | | 95% | |
|---|---|---|---|---|---|
| BMI SDS | n/N | vs. Control | aRR | CI | p-value |
| >1.65 | 66/484 | 22 [9.2%] vs | 0.51 | 0.31, 0.82 | 0.006 |
| | | 44 [18.0%] | | | |

Data are aRR and respective 95% CI from logistic regression adjusted for study site, infant sex, parity (multiparous/nulliparous), maternal smoking during pregnancy (none/active or passive), maternal pre-pregnancy BMI, and gestational age at birth.

### Example 4: Rapid Weight Gain and Increased Body Fat % (% FM)

From example 3, the maternal intervention was associated with a lower incidence of rapid weight gain (>0.67 SD from birth to 1 year; >1.34 SD from birth to 2 years). Early rapid weight gain is associated with a risk of later obesity. (Zheng M, et al. Obes Rev. 2018; 19 (3): 321-32). Because of this, we also assessed potential differences in body composition between children that experienced rapid weight gain vs. those that did not, irrespective of the treatment that their mothers received (i.e., adjusting for group and other factors). Similarly, we explored the differences in body composition between obese (BMI >95^{th} percentile) and non-obese children (BMI≤95^{th} percentile) at 2 years.

### Methods

Anthropometry: Birthweight was collected from hospital records, and subsequent weight measurements were measured to the closest 1 g until 1 year of age. At 2 years of age, weight was measured to the closest 0.1 kg. Crown-heel length was measured with a neonatometer or infantometer to 1 year of age. At 2 years old, standing height was measured using a stadiometer.

Bioelectrical impedance spectroscopy (BIS) measurements were taken at 6 weeks, 6 months, 1 year, and 2 years of age. The measurements were conducted using an ImpediMed SFB7 device (ImpediMed in Brisbane, Australia)

We used prediction equations specific to our cohort at 6 weeks and 6 months using PEA POD measurements (Lyons-Reid J et al. Prediction of fat-free mass in a multi-ethnic cohort of infants using bioelectrical impedance: Validation against the PEA POD. Frontiers in Nutrition. 2022; 9:980790):

$$6 - week males : FFM = 0.23 + 0.65 W + 0.10 {L}^{2} / {R}_{50}$$

$$6 - week females : FFM = 0.6 + 0.58 W + 0.08 {L}^{2} / {R}_{50}$$

$$6 - month males : FFM = 1.26 + 0.46 W + 0.14 {L}^{2} / {R}_{50}$$

$$6 - month females : FFM = 1.4 + 0.36 W + 0.21 {L}^{2} / {R}_{50}$$

Abbreviations: W, scale weight (kg); L, length (cm); R₅₀, resistance at 50 KHz. FM (kg) was calculated as the difference between measured weight and estimated FFM, with fat mass percentage (% FM) subsequently being calculated:

$$F M = W - FFM$$

$$\% F M = \{ F M / \{ F M + F F M ) ) * 100$$

At 1 and 2 years of age, the same 6-months equations were used.

### Results

Data on rapid weight gain from birth to 1 year and birth to 2 years is shown in table 2.

Regardless of the intervention effects, those who developed rapid weight gain (>0.67 SD) during the first year of life had similar body composition at 6 weeks but greater % FM at 6 months, 1 year, and 2 years of age (+2.0, +2.0, and +1.4 percentage points, respectively) compared to those who did not experience rapid weight gain. Children who experienced rapid weight gain >1.34 SD from birth to 2 years of age had lower % FM at 6 weeks (−0.9 percentage points) but increased % FM at subsequent visits (+1.4, +1.9, and +1.7, percentage points respectively).

Results showed that irrespective of the intervention effects, children who experienced rapid infant or early childhood weight gain or high BMI at 2 years old, developed higher levels of adiposity (% FM). Altogether, examples 3 and 4 demonstrate that more control group children experienced rapid weight gain and high BMI, which was characterised by greater % FM.

In conclusion, the intervention was associated with a reduction in the incidence of rapid weight gain in infancy and early childhood as well as high BMI at 2 years. Since children in these high-risk subgroups had greater levels of adiposity, the nutritional intervention can reduce the risk of experiencing excess fat mass.

1. A method of prevention of accelerated growth in an offspring, the method comprising administering a composition to the mother of the offspring during at least one of preconception, pregnancy or lactation, the composition comprising myo-inositol, a combination of Lactobacillus and Bifidobacterium probiotics, and at least one vitamin selected from the group consisting of vitamin B2, vitamin B6, vitamin B12, vitamin D and mixtures thereof.

2. The method of claim 1, which reduces risk of experiencing excess fat mass later in life.

3. The method of claim 1, wherein the Lactobacillus is the Lactobacillus rhamnosus strain available under the deposit number CGMCC 1.3724, and the Bifidobacterium is the Bifidobacterium lactis BB12 strain deposited as CNCM I-3446.

4. The method of claim 1, wherein the accelerated growth occurs during the first 2 to 5 years of life of the offspring.

5. The method of claim 3, wherein the at least one vitamin is vitamin B2, vitamin B6, vitamin B12, and vitamin D.

6. The method of claim 5, wherein the composition is administered in a daily dose that comprises from 0.2 to 5 g of the myo-inositol, from 0.14 to 14 mg of the vitamin B2, from 0.19 to 19 mg of the vitamin B6, from 0.26 to 26 μg of the vitamin B12, from 1.5 to 100 μg of the vitamin D, from 10⁵ to 10¹² cfu of the Lactobacillus rhamnosus CGMCC 1.3724 and from 10⁵ to 10¹² cfu of the Bifidobacterium lactis BB12 CNCMI-3446.

7. The method of claim 1, wherein the composition further comprises zinc.

8. The method of claim 6, wherein the composition comprises the zinc in an amount from 1.1 to 40 mg per daily dose.

9. The method of claim 1, wherein by preventing the accelerated growth, the composition further prevents development of metabolic disorders later in life in the offspring.

10. The method of claim 9, wherein the metabolic disorders are cardiometabolic disorders, obesity, excess fat mass deposition and/or overweight.

11. The method of claim 1, wherein the mother of the offspring is a mammal.

12. The method of claim 1, wherein the composition is administered to the mother of the offspring during pregnancy.

13. The method of claim 1, wherein the composition is in the form of a powdered nutritional composition to be reconstituted in milk or water, a food product, a drink, a nutritional supplement or a nutraceutical.

14. A kit of parts comprising at least two physically separated compositions, each of the physically separated compositions comprising at least one ingredient selected from the group consisting of vitamin B2, vitamin B6, vitamin B12 and vitamin D, at least one of the physically separated compositions comprises myo-inositol, and at least one of the physically separated compositions comprises a combination of Lactobacillus and Bifidobacterium probiotics, for prevention of accelerated growth in an offspring, wherein the two physically separated compositions are administered to the mother of the offspring during at least one of preconception, pregnancy or lactation.

15. The method of claim 1, wherein the mother of the offspring is selected from the group consisting of a cat, a dog and a human.

16. The method of claim 1, wherein the composition is administered to the mother of the offspring throughout the second and third trimesters of pregnancy.
